(19) United States
(12) Patent Application Publication
(10) Pub. No.: US 2003/0211049 A1
(43) Pub. Date: Nov. 13, 2003

(54) Device and method for reducing halitosis associated with smoking

(76) Inventor: Ofer Gerassi, Ramat Hasharon (IL)

Correspondence Address: DR. MARK FRIEDMAN LTD., C/o Bill Polkinghorn, Discovery Dispatch, 9003 Florin Way, Upper Marlboro, MD, 20772, US

(21) Appl. No.: 10/140,984

(22) Filed: May 9, 2002

(51) Int. Cl. A61K 9/68; A61K 7/16; F23D 14/28

(52) U.S. Cl. 424/48; 424/49; 431/344

(57) ABSTRACT

A combination device for reduction of smoking related halitosis. The device includes a lighter with a fuel reservoir and means of ignition, and an attachment mechanism, the mechanism designed and constructed to facilitate attachment of a breath freshening aid to the lighter

### FIELD AND BACKGROUND OF THE INVENTION

The present invention relates to a device and method for reducing halitosis associated with smoking and, more particularly, to a combination of a lighter and a breath freshening aid into a single compact unit.

It is well established that smoking tobacco (cigarettes, pipe, cigar) contributes to bad breath and/or unsightly stains on the teeth.

As a result, many smokers use breath freshening aids such as gum, candies and sprays to reduce halitosis. However, this requires that the smoker make a conscious effort to carry a breath freshening aid as well as tobacco and a source of ignition. Often, these smokers find that they have “forgotten” the breath freshening aid, although they rarely forget their tobacco or source of ignition.

There is thus a widely recognized need for, and it would be highly advantageous to have, a device and method for reducing halitosis associated with smoking devoid of the above limitation.

### SUMMARY OF THE INVENTION

According to one aspect of the present invention there is provided a combination device. The device includes (a) a lighter including a fuel reservoir and means of ignition thereof and (b) a attachment mechanism, said mechanism designed and constructed to facilitate attachment of a breath freshening aid to said lighter.

According to further features in preferred embodiments of the invention described below, the breath freshening aid may be, for example, chewing gum, candy, a spray or drops.

According to still further features in the described preferred embodiments the breath freshening aid is supplied in a dispenser attachable to the lighter by the reversible attachment mechanism.

According to still further features in the described preferred embodiments the attachment mechanism is a permanent or a reversible attachment mechanism.

According to another aspect of the present invention there is provided a method of reducing halitosis and/or tobacco stains on teeth, the method including attaching a breath freshening or tooth whitening aid to a lighter.

According to still further features in the described preferred embodiments the attaching is permanent or reversible.

According to still further features in the described preferred embodiments the lighter may be a container holding matches.

The present invention successfully addresses the shortcomings of the presently known configurations by providing a device and method for reducing halitosis associated with smoking by combining a lighter and a breath freshening aid into a single compact unit

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a is a side view of a lighter equipped with an attachment mechanism according to the present invention;

FIG. 1b is a side view of dispenser containing a breath freshening aid equipped with a attachment mechanism according to the present invention; and

FIG. 1c is a side view showing assembly of components seen in FIGS. 1a and 1b by means of the attachment mechanism.

### DESCRIPTION OF THE PREFERRED EMBODIMENTS

The present invention is of a device and method for reducing halitosis associated with smoking and, more particularly, to a combination of a lighter and a breath freshening aid into a single compact unit which increases the likelihood that a smoker will have a breath freshening aid available each time they smoke.

Specifically, the present invention can be used to assure a ready supply of gum candy spray or drops by attaching that supply to a lighter or matches.

The principles and operation of a device and method for reducing halitosis associated with smoking according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

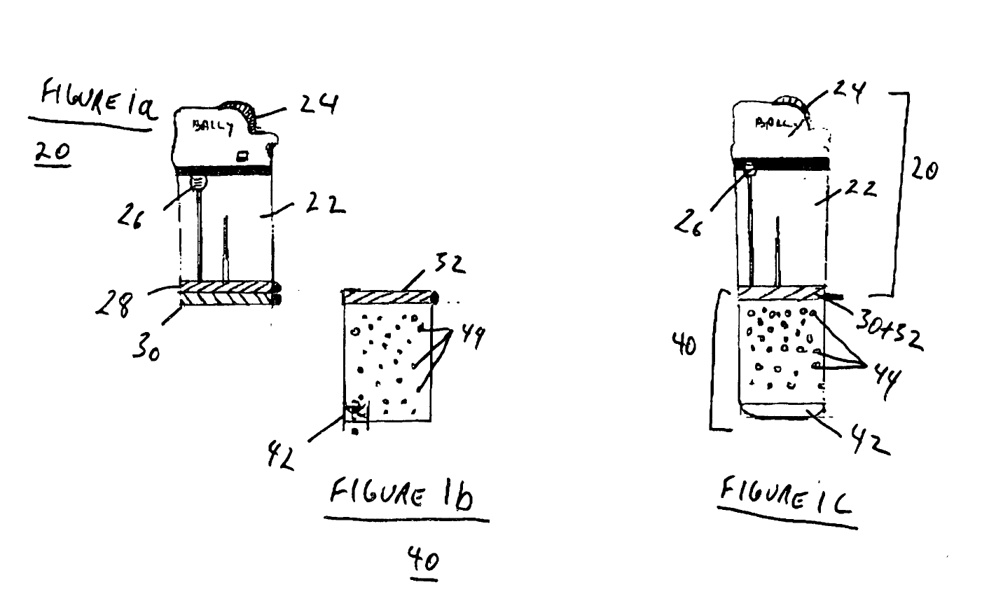

Referring now to the drawings, FIGS. 1a-c illustrate a combination device according to the present invention. The device includes a lighter 20 including a fuel reservoir 22 and means of ignition 24 and further including a t least a portion of a attachment mechanism 28, 30 designed and constructed to facilitate attachment of a container 40 of breath freshening aid 44 to lighter 20. Lighter 20 may include additional features such as a fuel supply regulator 26. Aid 44 may further contain a bleaching agent such as, for example, peroxide, to whiten teeth.

The breath freshening aid 44 may be, for example, chewing gum, candy, a spray or drops. The container 40 of aid 44 includes at least a portion of means of attachment 32 and a dispensing aperture or mechanism 42.

Thus breath freshening aid 44 may be supplied in a dispenser 40 attachable to lighter 20 by a reversible attachment mechanism 32 and 30.

Alternately the attachment mechanism 30 and 32 is a permanent attachment mechanism and the device is supplied as a single disposable unit.

Assembly of the device pictured in FIG. 1c constitutes practice of a method of reducing halitosis and/or tobacco stains on teeth. The method including attaching a breath freshening or tooth whitening aid 44 to a lighter 20. The attaching may be permanent or reversible.

Lighter 20 may be a container holding matches.

Additional objects, advantages, and novel features of the present invention will become apparent to one ordinarily skilled in the art upon examination of the following examples, which are not intended to be limiting.

Additionally, each of the various embodiments and aspects of the present invention as delineated hereinabove and as claimed in the claims section below finds experimental support in the following examples.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A combination device for reduction of smoking related halitosis, the device comprising:
(a) a lighter including a fuel reservoir and means of ignition thereof, and
(b) a attachment mechanism, said mechanism designed and constructed to facilitate attachment of a breath freshening aid to said lighter.

2. The device of claim 1, wherein said breath freshening aid is selected from the group consisting of chewing gum, candy, a spray and drops.

3. The device of claim 1, further comprising said breath freshening aid, wherein said breath freshening aid is supplied in a dispenser attachable to the lighter by said attachment mechanism.

4. The device of claim 3, wherein said attachment mechanism is selected from the group consisting of a permanent attachment mechanism and a reversible attachment mechanism.

5. A method of reducing halitosis and/or tobacco stains on teeth, the method including attaching an oral care product breath freshening or tooth whitening aid to a lighter.

6. The method of claim 5, wherein said oral care product includes at least one item selected from the group consisting of a breath freshening aid and a tooth whitening agent.
